(19) United States
(12) Patent Application Publication
(10) Pub. No.: US 2018/0055441 A1
(43) Pub. Date: Mar. 1, 2018

(54) ASSISTED CAPACITY DEVICE CALCULATOR (AC-DC)

(71) Applicant: KATRINA GOFF CANDY, Echuca (AU)

(72) Inventor: KATRINA GOFF CANDY, Echuca (AU)

(21) Appl. No.: 15/399,103

(22) Filed: Jan. 5, 2017

(30) Foreign Application Priority Data
Aug. 29, 2016 (AU) 2016903415

(51) Int. Cl. A61B 5/00; H04W 4/00; H04W 4/02; G06F 19/00; A61B 5/01; A61B 5/024; A61B 5/08; A61M 21/02

(52) U.S. Cl. CPC A61B 5/486; A61M 2021/0083; H04W 4/02; G06F 19/3406; A61B 5/681; A61B 5/6898; A61B 5/0002; A61B 5/4866; A61B 5/742; A61B 5/7455; A61B 5/01; A61B 5/02438; A61B 5/0816; A61B 5/749; A61B 5/746; A61M 21/02; H04W 4/008

(57) ABSTRACT

The Assisted Capacity Device Calculator (AC-DC) is an aide apparatus to help people manage their health by, among other things, totaling their daily intake of food and nutrients in their body by calculating weight, water intake, and waste while also calculating their total calorie expenditure. The apparatus is further constructed to identify the various factors that increase a person's body weight resulting from imbalances between caloric energy intake from food and caloric energy expenditure thru exercise, body heat, metabolic rate, etc.

### CROSS-REFERENCE TO RELATED APPLICATIONS

This application is a continuation of Australian provisional application 2016903415 of Katrina Goff Candy filed Aug. 29, 2016.

### FEDERALLY SPONSORED RESEARCH AND DEVELOPMENT

None.

### APPENDICES

None.

### BACKGROUND OF THE INVENTION

These is a need for an apparatus which has the capability to assist individuals in monitoring their health by determining how much food, exercise and rest they are getting on a daily basis. All of these factors affect an individual's weight and a healthy diet and exercise routine will both ensure proper weight gain or loss by preventing overeating and getting proper exercise. And as everyone is unique in terms of age, body type, body mass index, i.e., the ratio of fat to overall weight, metabolic rates and activity levels, homeostatic balances, etc., the number of calories a person ingests in the way of food and expends in calories, in the form of normal body function and exercise on a daily basis will vary. Additionally, appropriate water and hydration intake can vary by individual and can also be a critical element in attaining and maintaining a healthy body. Therefore, eating the proper amount and types of food versus the kinds and amounts of exercise one incurs daily will have a critical impact on one's weight and overall health.

### SUMMARY OF THE INVENTION

The AC/DC apparatus is constructed to monitor basic body parameters, via sensors incorporated in the AC/DC apparatus, such as body temperature, heart rate, respiratory rate, along with blood pressure and fluid values such as red/white body cell counts and glucose levels which are updated in the using software computer applications imbedded in the AC/DC apparatus, by micro-chip technology. Additionally, the AC/DC apparatus incorporates an electronic pedometer capable of measuring the user activity level such as distance traveled, intensity level, height or altitude, etc. and measures total calories expended at any given time.

The AC/DC apparatus in many ways replicates human body homeostats. Homeostats, in the body, are types of negative biological feedback mechanisms in the body used to regulate various body systems. For example, to regulate and maintain the body at 98.6 degrees Fahrenheit, the normal body temperature, a negative feedback loop in the form of your skin nerve cells sends a message to your brain if you the temperature goes above the normal body temperature. The brain subsequently sends a message to sweat glands to open and release water thereby bringing the body temperature down to the normal temperature. The AC/DC apparatus is further constructed to be programmed with the user's metabolic analysis and various homeostatic analyses including body core temperature homeostat along with blood related measures such as blood glucose and oxygen content homeostat, blood partial pressure of oxygen/carbon dioxide homeostat, blood oxygen content homeostat, blood arterial blood pressure homeostats as well as plasma ionized calcium homeostat. Additionally, it is also critical to assess the user's extracellular sodium and potassium concentration along with volume of body water and extracellular fluid pH homeostats.

Among other things, the AC/DC apparatus is constructed to record, monitor and show a user their daily food intake and make suggestions to the user regarding healthy food choices. For example, when one is planning a meal, choosing a snack reading food labels is critical in determining proper food choices. Additionally, the amount and type of daily ingested nutrients, be it macro nutrients, i.e. water, protein, carbohydrates, fats, fiber, or micro nutrients, i.e., vitamins and minerals, must be carefully analyzed and recorded by the user. And as nutrients generally provide material for assisting and maintaining cellular growth, providing body energy, in the form of calories, and other for body functions.

Energy for the body is generated primarily by proteins and carbohydrates which during the metabolic process produces sugars which are the main source for energy in the human body. Other sugars, usually in the form of fructose, naturally occurring sugar in fruit for example, are also a major source of energy for human. For many individuals, foods containing added sugar, many times in the form of sucrose, versus naturally occurring sugar are particularly problematic for attaining and maintaining proper body weight. Reducing and controlling the amount of added sugar is a major approach of preventing diseases such as diabetes.

The AC/DC apparatus also has a copy of the user's medical records and genetic genome to allow for instant access to the user total medical and genetic history especially in emergency medical situations.

In addition to nutrition, another critical factor in attaining and maintaining body weight and health is a regular exercise routine. This may involve simple household chores such a cooking, cleaning, etc. but may also include dedicated routines such as walking, running, cycling, gym or health center regiments. Finally, a major component to well-being includes sufficient among of sleep which for children is particularly essential to ensure growth and health while in adults the proper among of sleep minimizes various health problems. The AC/DC apparatus pedometer is capable of measuring these activities and the AC/DC apparatus software is constructed to calculate and monitor the user's daily calorie expenditure.

Other factors that may affect an individual's weight and overall health is the amount of muscle mass produced by weight training, medication, disease and for women natural conditions such menopause or pregnancy. Additionally, the AC/DC apparatus is constructed to monitor other physical body attributes such a walking gait and posture and to alert the user in event of changes in those attributes.

The instant invention comprises an Assisted Capacity Device Calculator (AC-DC), hereinafter “AC/DC apparatus”, and system to assist and help people with overall health by recording and/or monitoring food intake and exercise for a user.

As a typical example for use the AC/DC apparatus, the user would be assessed or have an assessment conducted to determine age, BMI, weight, height, etc. along with a biochemical/physiological evaluation as part of an overall health profile including vital signs, i.e., heart rate and pressure, respiratory rates, etc. A critical factor to be assessed is a person's resting metabolic rate. Additionally, there would be a biochemical/physiological evaluation, among other things, which would measure a person's white blood count, hemoglobin, hematocrit and platelet count along with sodium, potassium and fasting glucose, triglyceride, cholesterol, and liver enzymes levels, etc. These evaluations are measured against normal ranges for similar individuals of age, gender, etc. Subsequently, a regiment would be developed by the AC/DC apparatus for the user for food intake and exercise daily and the as the user progresses during the day the of day the AC/DC apparatus monitors the user. Any adjustment of food and or other activities for the user would then be calculated and provided to the user to make appropriate adjustments. As human beings are endotherms in the animal kingdom, that is their bodies have metabolic rates which produce sufficient energy to heat up and maintain their body temperature at a relative constant temperature, usually 98.6 degrees Fahrenheit. For male humans, on average, their bodies require between 1600 to 1800 kilocalories of energy per day to function normally while female humans require between 1300 to 1500 kilocalories of energy per day. These are daily requirements for human at rest; obviously the more active an individual, the greater amount of energy is required.

The AC/DC apparatus is further constructed to either be mobile or stationary and may come in various applications, e. g, such as a cord necklace, or lanyard fitted AC/DC apparatus, to be worn by the user. The AC/DC apparatus is also constructed to be fitted to a wrist bracelet or watch band and if further provided with a camera intake/scanner feature for scanning food or other labels to determine caloric and other nutrition information.

### BRIEF DESCRIPTION OF THE DRAWINGS

FIG. 1 discloses a first embodiment of the AC/DC apparatus.

FIG. 2 is a detailed figure of the AC/DC apparatus 2 and its operation with either a mobile electronic tablet and/or a mobile electronic smart phone device.

FIG. 3 is a detailed figure of the AC/DC apparatus of FIGS. 1 and 2 showing other features of AC/DC apparatus 2.

FIG. 4 is a detailed figure of the AC/DC apparatus 2 of FIGS. 1-3 disclosing its microchip and power source.

FIG. 5 discloses a second embodiment of the AC/DC apparatus.

FIG. 6 shows details of the AC/DC apparatus of FIG. 5.

FIG. 7 shows further details of the AC/DC apparatus of FIG. 5.

FIG. 8 shows further details of the AC/DC apparatus of FIG. 5

FIG. 9 shows a third embodiment of the AC/DC apparatus.

FIG. 10 shows further details of the AC/DC apparatus of FIG. 9.

FIG. 11 is a task flow chart showing how the AC/DC apparatus and system function to perform its operation.

FIG. 12 is an example of how an energy homeostat operates in a user body.

FIG. 13 is a step by step flow chart of an example of the operation of the AC/CD apparatus.

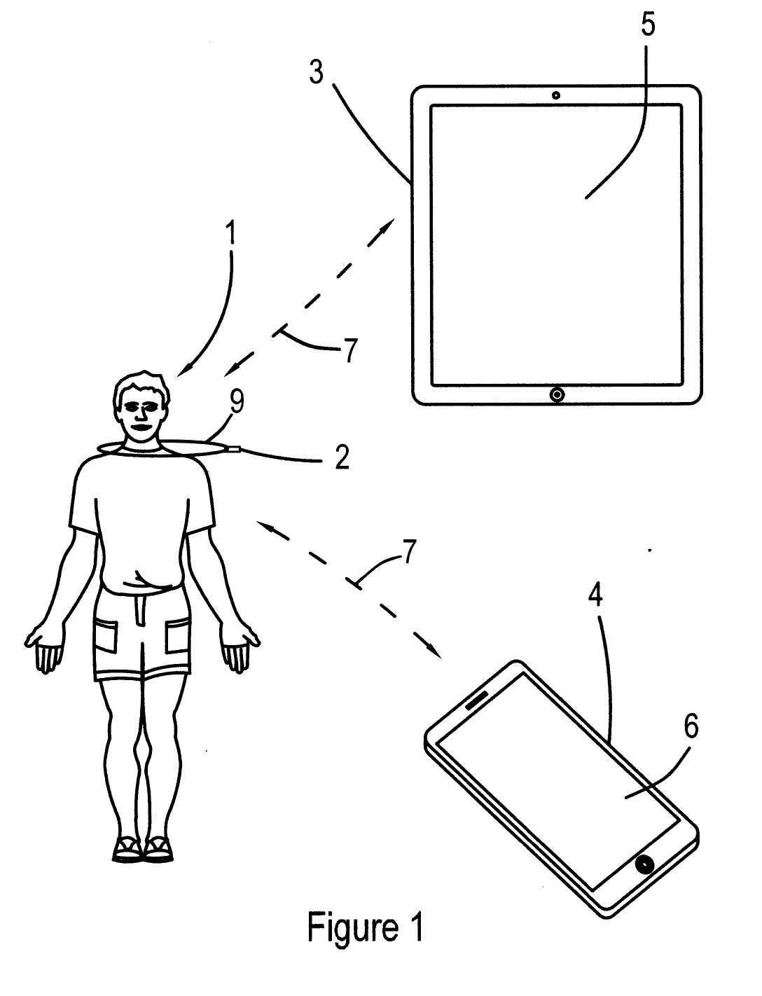

FIG. 1 shows a first embodiment of the AC/DC apparatus particularly AC/DC apparatus 2 being utilized by a user 1. In FIG. 1, the AC/DC apparatus 2 is shown being attached to user 1 via lanyard or necklace 9 and the AC/DC apparatus 2 is capable of electronically communicating, by wireless signals 7, as is disclosed and discussed below, with an electronic appliance in the form of an electronic tablet 3 having a visual screen 5 and/or smart phone device 4 and having visual screen 6.

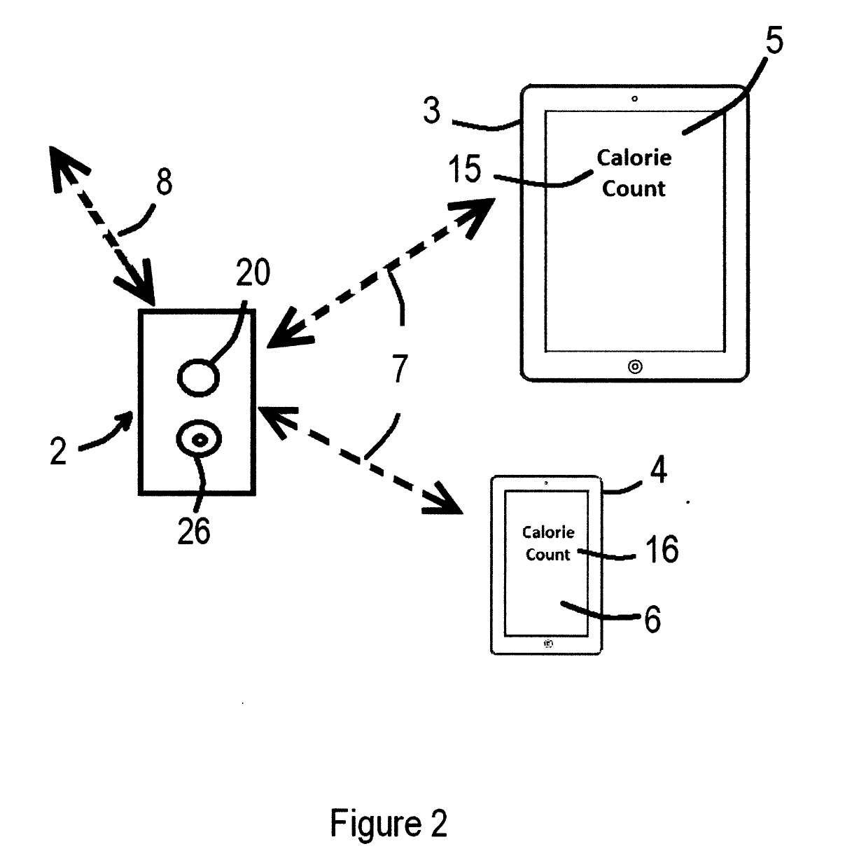

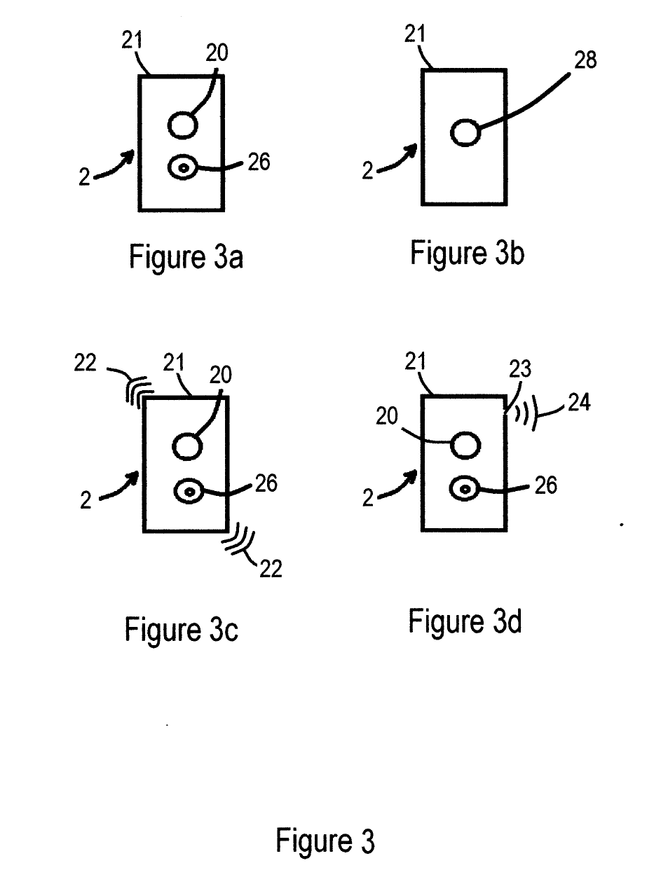

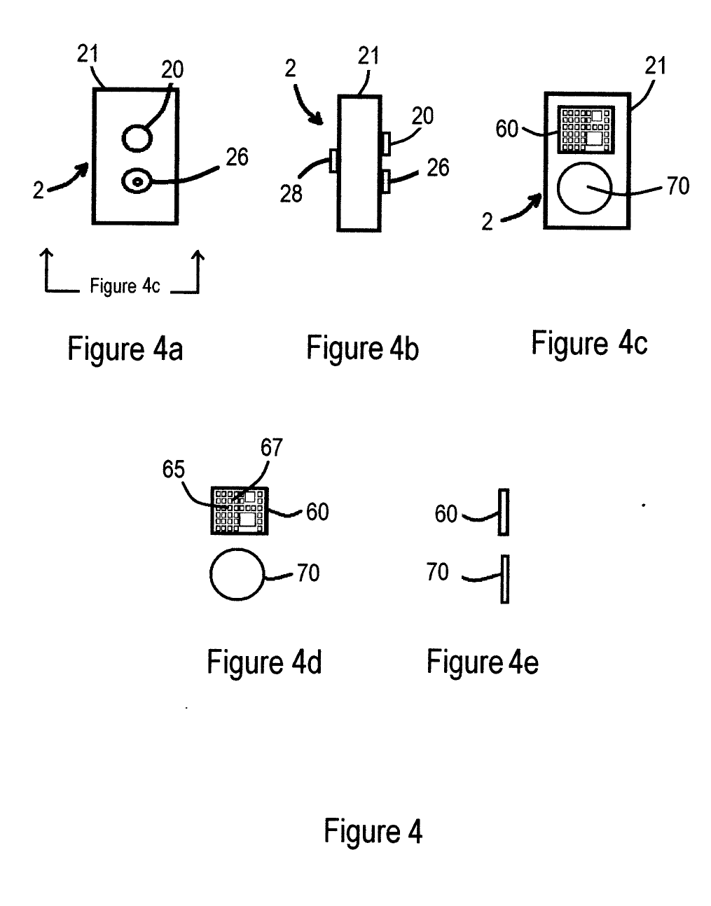

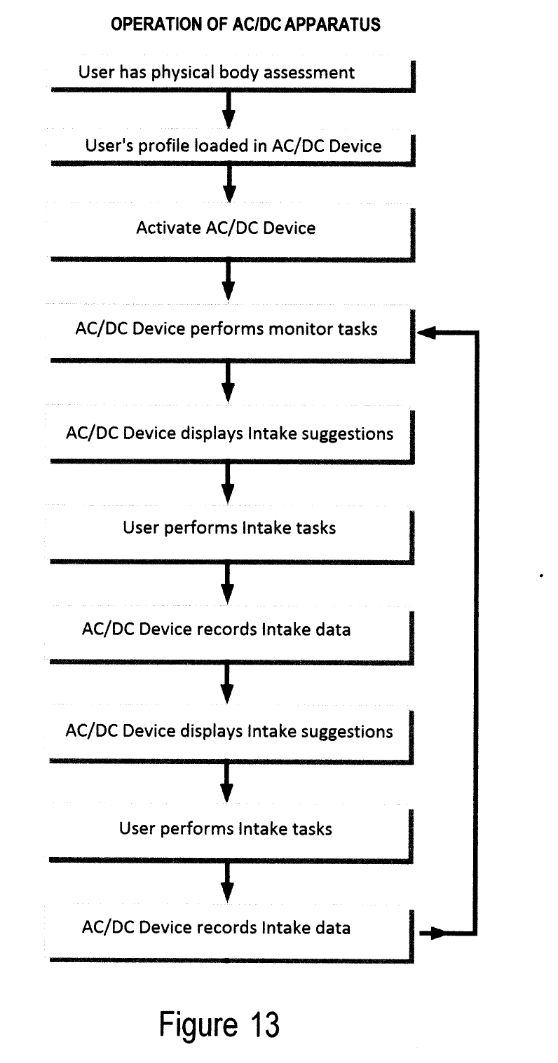

FIG. 2 discloses further details and operation of the AC/DC apparatus 2, particularly, FIG. 2 shows that AC/DC apparatus 2 is provided with a button 20 which can be depressed by the user to activate AC/DC apparatus 2. Additionally, AC/DC apparatus 2 is capable of creating transmitting and receiving electronic signals 7, via a wireless signal transmitter imbedded in AC/DC apparatus 2 in the form of a microcomputer sensor transceiver processor 60, further disclosed and discussed in reference to FIG. 4. Upon activation, microcomputer sensor transceiver processor 60 sends a wireless signal 7, by various systems, such as a technology that allows electronic devices to network, i.e., WI-FI, or a wireless technology standard for exchanging data over short distances commonly known as Bluetooth, or ZigBee, etc. Additionally, wireless signal 7 can be transmitted to an emergency alert system such medical hospital or clinic alert staff desk, nursing station, emergency office or other medical facility. The wireless signal 7 is also transmittable to a mobile electronic appliance, such as an electronic tablet 3 or a smart phone 4, also shown in FIG. 2. The mobile electronic appliance, either electronic tablet 3 or smart phone 4 is capable of sending, receiving, and processing the wireless signals 7 and/or data and also is compatible with the Android mobile operating system, the iPhone cellular phone mobile operating system, i.e., Ios, and other commercially available mobile device operating systems. Additionally, the microcomputer sensor transceiver processor 60 incorporates radiofrequency microchip technology, discussed further in reference to FIG. 4, capable of sending, receiving, and processing data and/or signals 8 to and from navigational positioning systems, such as the Global Positioning System, i.e., GPS, or the Global Navigational Satellite System, i.e., the GLONASS for geographically locating the microcomputer sensor transceiver processor 60. Microcomputer sensor transceiver processor 60 is further capable of being coded with information, in a software application, otherwise call an app, 65, shown in FIG. 4d, regarding the identity of the user such as his or her birth date, home location, medical records, and other the medical information of the user. The medical information may include, as discussed further in regards to FIG. 13, a physical body assessment. The AC/DC apparatus 2 further incorporates a pedometer 67, in microcomputer sensor transceiver processor 60 as further discussed in reference to FIG. 4d, in the form of a MEMS, i.e., a microelectromechanical system in the form of an inertial sensor capable of measuring 1 to 3 axis acceleration data, which data is processed by the app 65; the data from is capable of calculating expended User calorie levels based on the pedometer sensor data. App 65 is further programmed to receive input from the user and from data from the pedometer and sensor probe 28, discussed further in reference to FIG. 3b, and co-ordinates with app 5 of electronic tablet 3 and/or app 6 of electronic smartphone 6, to make various calculations regarding the user's medical and physical condition particularly caloric intake and expenditure. The AC/DC apparatus is also provided with a camera scanner 26 allowing the apparatus to either take and record pictures and photos or to scan bar codes particularly bar codes on food packages for inputting calorie and nutrition information into AC/DC apparatus 2. Finally, the AC/DC apparatus 2 is programmed to be operated by either an app, not shown, of electronic tablet 3 and/or app, not shown, of electronic smartphone 6 to display or readout data of various functions of the AC/DC apparatus 2, particularly to the measure heart rate, respiratory rate or temperature of a user.

As is further shown in FIG. 2, the mobile electronic appliance, either electronic tablet 3 or smart phone 4, is provided with an app, not shown, which is capable of processing any signal and/or data received by a sensor device from the AC/DC apparatus 2 and also capable of producing a readout or display on a visual screen display. For the electronic tablet 3, this readout or display is shown as reference number 15 on visual screen 5 while for the smart phone 4, the readout or display is shown as reference number 16 on visual screen 6. In the example of FIG. 2, the readout or display is “Calorie Count” and refers to have the AC/DC apparatus 2 performing particular physical body attribute task, here to calculate the total number of calories consumed by the user and the total number of calories expended by the user for a net total calculation. The calorie count can also display the types of calories consumed as well as the type and how the calories have been expended. Additionally, electronic tablet 3 and smart phone 4 are touch activated by the user to perform the various tasks desired by the User.

As previously discussed in reference to FIG. 2, camera scanner 26 allows the user to scan food labels to input calorie and nutrition information into the apparatus. Although the example given in FIGS. 1 & 2 of the physical body attribute calculation is related to caloric intake, AC/DC apparatus 2 is capable of determining numerous physical body attributes, via sensor probe 28, as further discussed in reference to FIG. 4 above, such as body temperature, heart and respiratory rates. The AC/DC apparatus 2 is disclosed, as is discussed in reference to FIG. 4, as being powered by a battery, although the apparatus is can be configured to be with solar energy.

FIG. 3 reveals additional features of AC/DC apparatus 2. Particularly FIG. 3a shows the overall construction of AC/DC apparatus 2 having a body 21 and an activation button 20 and a camera scanner 26. FIG. 3b is a rear view of FIG. 3a revealing sensor probe 28 capable of, among other things, sensing a user's temperature, body heart pulse rate and/or respiratory rate. FIG. 3c reveals the vibration feature 22 of AC/DC apparatus 2 allowing the apparatus to act in an alarm or awakening mode while FIG. 3d reveals a sound transmitter and receiver 23 provided with AC/DC apparatus 2 capable of producing alarm or awakening sound 24. Sound transmitter and receiver 23 is also capable of receiving voice commands or input for operating the AC/DC apparatus 2.

FIG. 4 is a detailed figure of AC/DC apparatus 2 showing details of the apparatus construction. Particularly, FIG. 4a discloses that AC/DC apparatus 2 has a body 21 and activation button 20 and camera scanner feature 26. FIG. 4b is a side view of FIG. 4a showing AC/DC apparatus 2 in profile showing activation button 20, camera scanner 26, and sensor probe 28. As discussed in reference to FIG. 3b sensor probe 28 is constructed to measure, inter alia, either a user's temperature, heart or respiratory rate. FIG. 4c is a cross-sectional view of FIG. 4a showing internal operational components of AC/DC apparatus 2, particularly microcomputer sensor transceiver processor 60 and power source 70 in the form of a disc battery. FIG. 4d shows the microcomputer sensor transceiver processor 60 and power source 70 individually revealing their relative size and imbedded app 65 capable of processing sensor probe 28 data as well as other input data. FIG. 4e is a side profile view of microcomputer sensor transceiver processor 60 and power source 70. As previously discussed, microcomputer sensor transmitter processor 60 is capable, via app 65, of being coded with identity, medical and physical body assessment information regarding the user, as discussed above, and is capable of creating and receiving signals and/or data which can be transmitted to a satellite navigational technology system as discussed in reference to FIG. 2, supra. Microcomputer sensor transceiver processor 60 also can transmit and receive signals and/or data to the mobile electronic appliance, electronic tablet 3 or smart phone 5 and is programmed with app 65 for analyzing and processing medical and physical body input data such a heart rate, body temperature or respiratory rates.

As previously discussed, microcomputer sensor transmitter processor 60 is capable, via app 65, of being coded with identity, medical and physical body assessment information regarding the user, as discussed above, and is capable of creating and receiving signals and/or data which can be transmitted to a satellite navigational technology system as discussed in reference to FIG. 2, supra. Microcomputer sensor transceiver processor 60 also can transmit and receive signals and/or data to the mobile electronic appliance, electronic tablet 3 or smart phone 5 and is programmed with app 65 for analyzing and processing medical and physical body input data such a heart rate, body temperature or respiratory rates. Additionally, as previously discussed, and shown further in reference to

FIG. 4d, microcomputer sensor transceiver processor 60 incorporates a MEMS inertial sensor capable of measuring 1 to 3 axis acceleration, i.e., a pedometer 67, data from which is processed by app 65 of calculating input and expended calorie levels of the user.

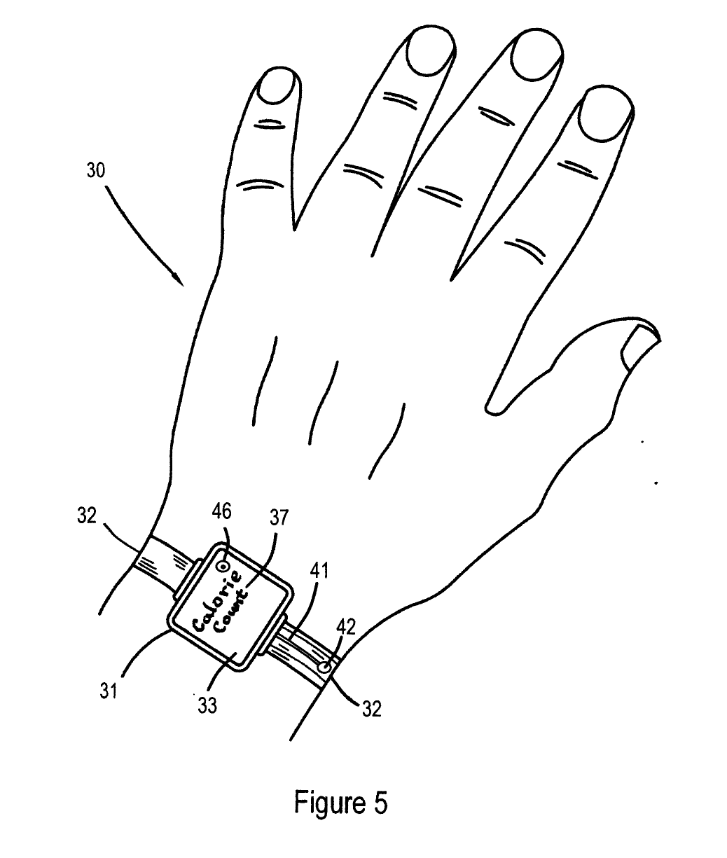

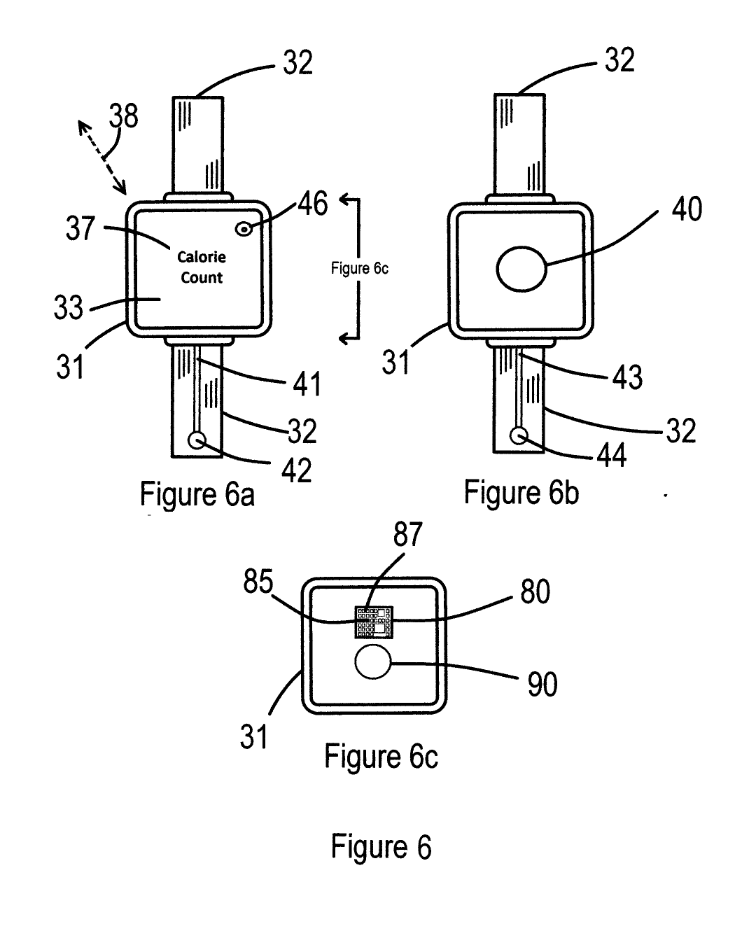

FIG. 5 discloses a second embodiment of the AC-DC apparatus. Particularly, FIG. 5 discloses a AC-DC apparatus 31 housed in the form of watch type configuration, having watchband 32, being worn on a hand 30 of the user 1. Additionally, as FIG. 5 discloses, AC-DC apparatus 31 has a visual screen 33 and visual screen display readout 37, produced via an app 85, as shown and discussed in reference to FIG. 6c, imbedded in the apparatus 31 similar to that of AC-DC apparatus 2 app 65. The visual screen display readout 37 is capable of displaying or showing a readout to the user 1, here, as an example

“Calorie Count”. AC-DC apparatus 31 is further configured to be touch activated on its screen by the user and can received input from the user. AC-DC apparatus 31 can also be configured with traditional push buttons, not shown. Additionally, AC-DC apparatus 31 has an illumination display feature allowing the apparatus to be seen at night or in low-light environments. Also, seen in FIG. 5, is camera scanner 46 allowing scanning of food and dietary information, for input calorie and nutrition information similar to camera scanner 26 of AC-DC apparatus 2. Further shown in FIG. 5, is a sensor probe 42, similar to sensor probe 28 of AC-DC apparatus 2, which sensor probe 42 is also capable of, inter alia, sensing a user's body temperature, heart pulse rate or respiratory rate. Sensor probe 42 is connected to AC-DC apparatus 31 by electrical connector 41. All of the data from sensor probe 42 is capable of being inputted into an app 85 located in microcomputer sensor transceiver processor 80, shown and further discussed in reference to FIG. 6c. Additionally, AC-DC apparatus 31 and microcomputer sensor transceiver processor 80 also incorporates a MEMS, i.e., a microelectromechanical system in the form of inertial sensor capable of measuring 1 to 3 axis acceleration, i.e., a pedometer 87, shown and discussed in reference to FIG. 6c. App 85 is functionally similar to app 65 of AC-DC apparatus 2 and also programmed to receive input from the user at any time, via and from data from the pedometer 87 and sensor probes 40, 42, or 44, as shown and discussed in reference to FIG. 6, to make various calculations regarding the user's medical and physical condition particularly caloric intake and expenditure.

FIG. 6 shows details of the AC-DC apparatus 31 of FIG. 5. Particularly, FIG. 6a shows AC-DC apparatus 31 with wristband 32, visual screen 33, visual screen display readout 37, and particularly reveals the wireless communication signal feature producing and receiving electronic signals 38 from microcomputer sensor transceiver processor 80 of FIG. 6c. FIG. 6b, a rear view of AC-DC apparatus 31 of FIG. 6a, shows sensor probe 40 and sensor probe 44. Sensor probe 42 is located and positioned on band 32 such that can be held against the chest area of a user to measure the user's respiratory rate while sensor probe 44 is constructed and located on the AC-DC apparatus 31 to sense the heart rate on the wrist of a user. Sensor probe 44 is further capable of sensing a user's body temperature. FIG. 6c, a cross-section view of AC-DC apparatus 31 of FIG. 6a, reveals that AC-DC apparatus is internally provided with electronic components 80 and 90 similar to microcomputer sensor transceiver processor 60 and power source 70 of AC-DC apparatus 2. As such, AC-DC apparatus 31 is capable of electronically communicating with GPS and GLONASS satellite systems for allowing location of wearer of the apparatus, i.e., the user. As with AC-DC apparatus 2, and as discussed supra, in reference to FIG. 5, microcomputer sensor transceiver processor 80 of AC-DC apparatus 31 incorporates a

MEMS inertial sensor capable of measuring 1 to 3 axis acceleration, i.e., a pedometer 87, which is processed by imbedded software app 85 capable of calculating calorie levels or other calculations as previously discussed, supra.

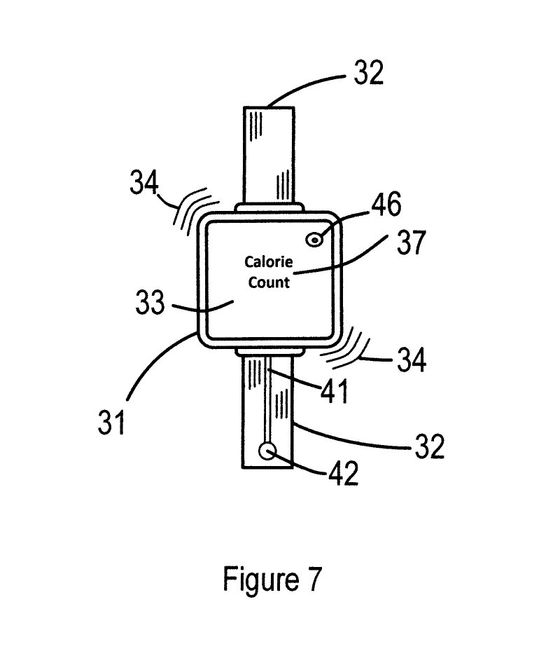

FIG. 7 discloses an additional feature of the AC-DC apparatus 31 of FIG. 5, particularly, a vibration capability 34 allowing apparatus 31 to remind and or alert the user to perform or to display a particular visual display readout, here, as an example, to “Calorie Count”.

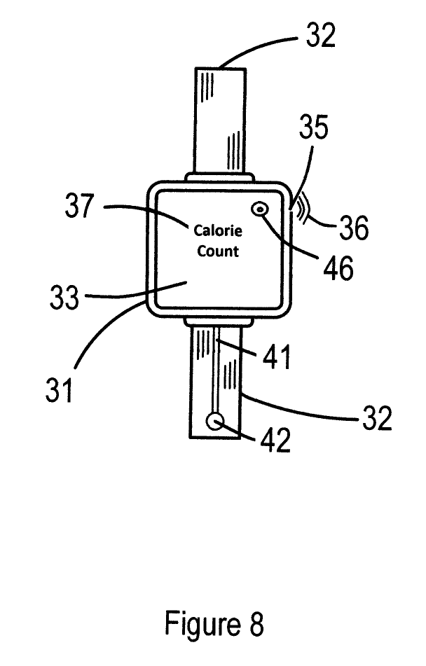

FIG. 8 discloses an additional feature of the AC-DC apparatus 31 of FIG. 5, particularly, a sound transmitter and receiver 35 capable of emitting sound 36 for alerting the user to remind and or alert the user to perform or to display a particular visual display readout, here, as an example, to “Calorie Count”. Additionally, transmitter and receiver 35 is capable of receiving audible input which can be used to program AC-DC apparatus 31.

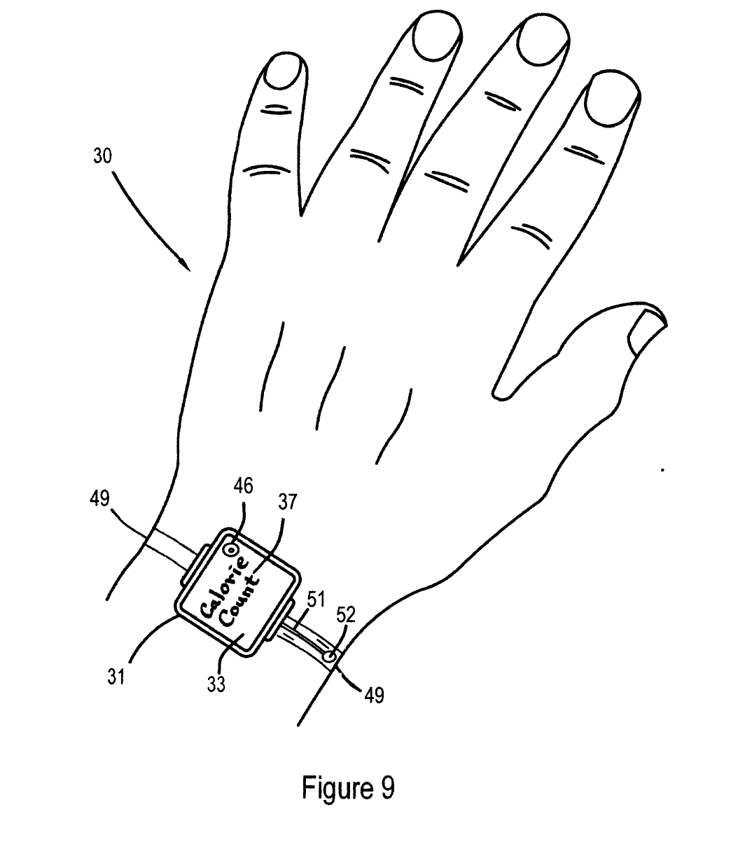

FIG. 9 shows a third embodiment of the AC-DC apparatus 31 being utilized by a user 1, consisting of a bracelet 49 securing AC-DC apparatus 31 on a hand 30 of the user 1, such that the AC-DC apparatus has a visual screen 33 and screen display 37 capable of communicating a readout to the User. The AC-DC apparatus 31 of FIG. 9 has all of the features and capabilities of the AC-DC apparatus 31 of FIGS. 5-8. Also seen in FIG. 9, the AC-DC apparatus 31 is provided with the camera scanner feature 46 along with sensor probe 52 which operates in a similar fashion to sensor probe 42 of FIGS. 5-8 and are provided with electronic components 80 and 90 as well as app 85 and pedometer, discussed in reference to FIG. 6c, capable of producing all of the electronic information and processing all of the data of the AC-DC apparatus 31 of FIG. 5.

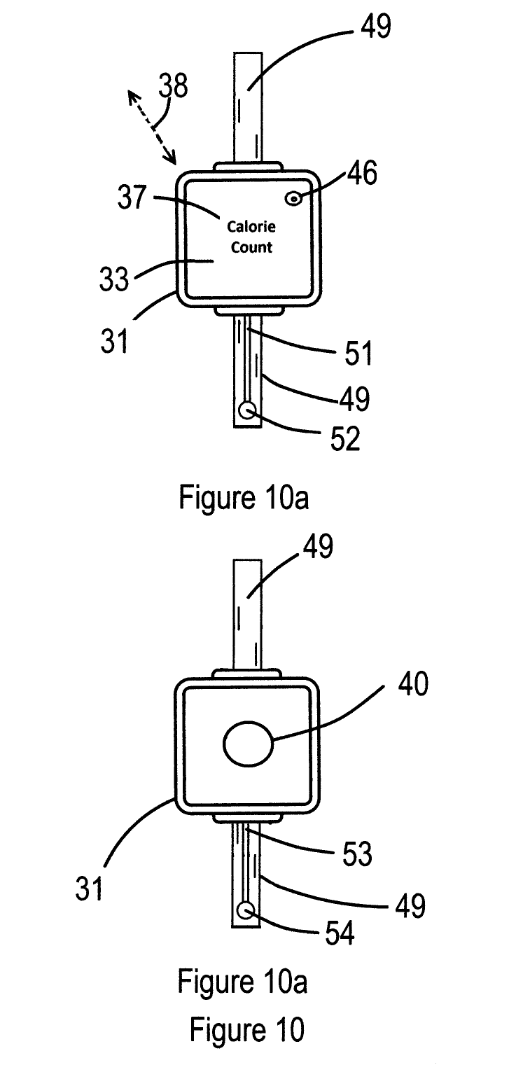

FIG. 10 shows details of the AC-DC apparatus 31 with bracelet 49. The AC-DC apparatus 31 of FIG. 10 has all the features of the AC-DC apparatus of FIG. 6; however the bracelet 49 differs from wristband 32 such that bracelet type device is thinner than wristband 32 as seen in FIG. 10a. FIG. 10a furthers reveals sensor probe 52 connected to AC-DC apparatus 31 by electrical connector 51. FIG. 10b is a rear view of the AC-DC 31 attached to bracelet 49 revealing sensor probe 54 attached to AC-DC apparatus 31 electrical connector 53. Sensor probes 52 and 54 function identically as sensor probes 42 and 44 of FIGS. 5 to 8 above and AC-DC apparatus 31 of FIG. 10 is provided with electronic components 80 and 90 as well as app 85 and pedometer, discussed in reference to FIG. 6c, capable of producing all of the electronic information and processing all of the data of the AC-DC apparatus 31 of FIG. 5.

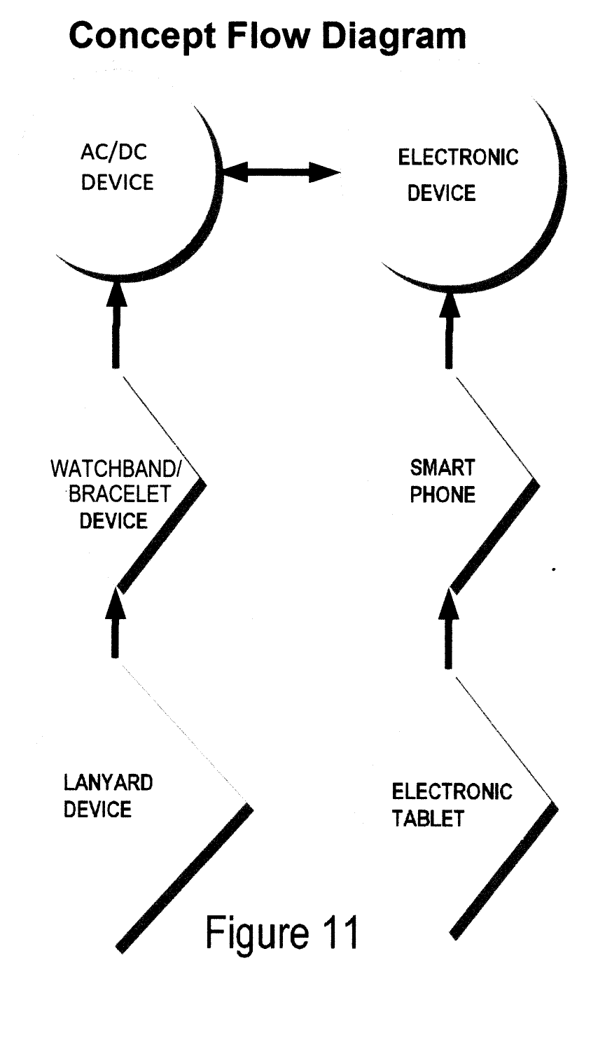

Now referring to FIG. 11, a concept flow diagram of the system according to the preferred embodiment of the invention is shown. Particularly in FIG. 10, the overall operation of the preferred embodiment of the invention is disclosed wherein the AC-DC apparatus, denoted by the encircled term apparatus may comprise wrist watchband, bracelet, lanyard attachment, etc. As FIG. 11 furthers discloses, the AC-DC apparatus and system, whether it is the embodiment of FIG. 1, 5 or 9, of the instant invention is provided with a sensor device capable of creating and transmitting electronic signals, via a wireless signal transmitter imbedded in the sensor device. The wireless signals are transmittable to a mobile electronic appliance, in the AC-DC apparatus 2, such as a smart phone, or an electronic tablet. The mobile electronic appliance is capable of sending, receiving, and processing the wireless signals and/or data and also is compatible with the Android mobile operating system, the iPhone cellular phone mobile operating system, i.e., Ios, and other commercially available mobile device operating systems. Additionally, the sensor device of the invention incorporates radiofrequency microchip technology, in the form of a microcomputer transmitter processor capable of sending, receiving, processing, and transmitting data and/or signals to and from navigational positioning systems, such as the Global Positioning System for locating the sensor devices which signals and/or data can transmit signals and/or data to the mobile electronic appliance. As is further shown in FIG. 10, the mobile electronic appliance is provided with or is accessible to computer processing application software, i.e., otherwise known as an app, which app is capable of processing the any signal and/or data receiving by the sensor device.

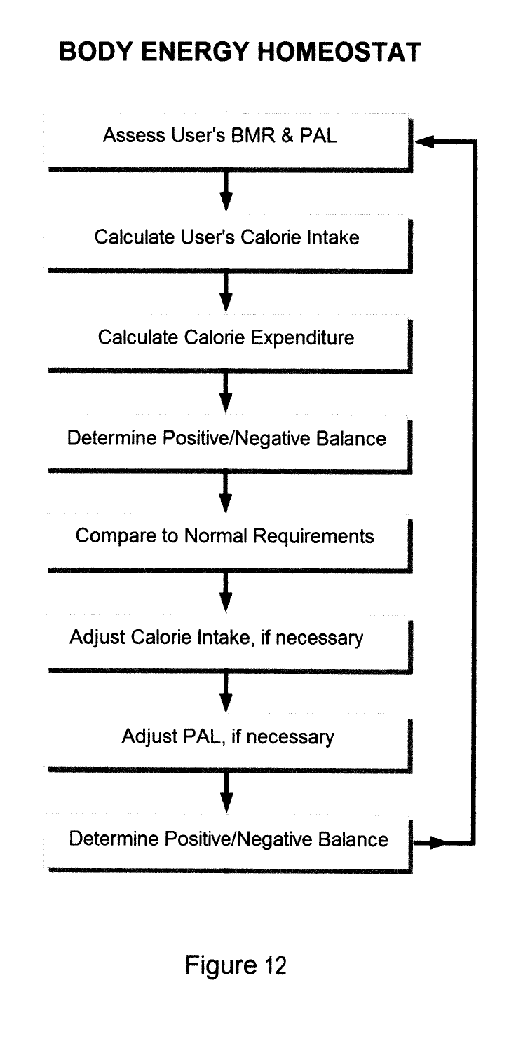

FIG. 12 is an example of how an energy homeostat functions in a User body. Particularly, FIG. 12 demonstrates there is a homeostat, in the human body for regulating the amount of energy intake and expenditure. Controlled primarily by the hypothalamus in the brain, which generates a sense of hunger, the body energy homeostat determines the amount of energy intake, in the form of ingested food and compares this to energy output based primarily on basal metabolic rate, otherwise known as BMR, along with the physical activity level, otherwise known as PAL. The brain then determines if there is an imbalance, a positive balance implying that the user is typically overeating or lacks sufficient physical activity, or a negative balance usually caused by undereating. In medical science, in order to advise users regarding the best diet and/or physical regiment, several methods are used to determine normal human body energy requirements, the most being popular probably being the Harris-Benedict equation. The AC-DC apparatus in many ways replicates the human body energy homeostat.

FIG. 13 shows a step by step operation of the AC-DC apparatus 2 or 31. The user initially will undergo a complete body physical and medical assessment involving body dimensions along with body mass index calculations, heart rates, blood pressure measurements and a body biochemical analysis including blood analyses, etc. to create a user health profile. The user's health profile is then loaded into the AC-DC apparatus for use on a daily basis, particularly in regards to caloric intake and expenditure. Upon activation for daily use, the AC-DC performs monitoring tasks and makes dietary intake suggestions for the user, in the form of food, beverage and/or water intake and suggested physical exercises. The user then concurs with and executes some or all of the suggestions which are then recorded as intake data, by either being inputted by the user directly, or by using scanner camera 26 or 46, in the case of food or beverage into and processed by the AC-DC apparatus. The pedometer 67 or 87 of either AC-DC apparatus 2 or 31 then sends data to apps 65 and/or 85 which calculates the expended calories for suggested exercises and displays the results on the of caloric intake and expenditure on either electronic tablet 3, electronic smartphone 6 or device 31.

In addition, at any time during a routine, the user can determine his/her body temperature by activating the AC-DC apparatus 2 or 31 and that data can be inputted into the apparatus. For the AC-DC apparatus 2, the resulting data calculation is displayed on either electronic appliance 3 or 4. In the example shown in FIG. 2, the AC-DC apparatus 2 has performed a calorie count calculation to determine total calories consumed and expended by the user and visually displayed “Calorie Count” on visual screen 5 or 6 of the respective electronic appliance. As a further example, the user may determine his or her heart rate by placing the AC-DC apparatus 2 sensor probe 28 against either the underside of his/her wrist or against the side of their neck where heart pulse is ordinarily measured. For the AC-DC apparatus 31, the user can measure the temperature or heart rate by activating the apparatus 3 and using sensor probe 44 to take the appropriate measurement. To measure a user's respiratory rate, for the AC-DC apparatus 2, the user places the sensor probe 28 against the appropriate chest area of the User while for the AC-DC apparatus 31, the user places the sensor probe 42 against the appropriate chest area of the user. All the inputted data is then processed by either app 65 for apparatus 2 or app 85 for apparatus 31. The AC-DC apparatus then makes additional intake or expenditure suggestions, if necessary, and the cycle of intake data until a daily intake routine is completed. The user will then modify his/her routine to complete a satisfactory expenditure level. Although this example describes the use of a AC-DC apparatus for a morning routine, it is to be understood, that the AC-DC apparatus can also be programmed for afternoon, evening, night, or any desired routine.

When introducing elements of the present invention or the preferred embodiment(s) thereof, the articles “a”, “an”, “the”, and “said” are intended to mean there are one or more of the elements. The terms “comprising”, “including”, and “having” are intended to be inclusive and mean that there may be additional elements other than the listed elements. Additionally, any description of the exemplary or preferred embodiments is intended to be read in connection with the accompanying drawings, which are to be considered part of the entire written description.

Additionally, the principles of the invention could be practiced by those of skilled in the art with equivalent alternative constructions. Although the present invention has been described in considerable detail with reference to a certain preferred embodiment thereof, other embodiments are possible. Therefore, the spirit and scope of the appended claims should not be limited to the description of the preferred embodiment(s) contained herein. The invention may be embodied and practiced in other specific forms without departing from the spirit and essential characteristics thereof. The present embodiments are therefore to be considered in all respects as illustrative and not restrictive, the scope of the invention being indicated by the appended claims rather than by the foregoing description; and all variations, substitutions and changes which come within the meaning and range of equivalency of the claims are therefore intended to be embraced therein.

What is claimed:

1: A wearable assisted capacity device apparatus for assisting a user in managing their health comprising
means for electronically communicating said apparatus with an electronic appliance and wherein the apparatus can be activated by the user and further wherein the apparatus is provided with
means which can send and receive electronic signals and further wherein the apparatus is provided with
means to produce a wireless signal which is transmittable to a mobile electronic appliance and further wherein said electronic appliance is provided with a computer processing application software which can process any signal received by the apparatus and further wherein the computer processing application software can produce a visual display on the electronic appliance to inform, remind or command the user to perform a certain task.

2: The wearable assisted capacity device apparatus of claim 1 further provided with means for transmitting to and receiving a wireless signal from a navigational positioning system.

3: The wearable of claim 2 further provided with means in the form of push button for activating the apparatus by the user.

4: The wearable assisted capacity device apparatus of claim 3 further provided with means for causing the apparatus to vibrate.

5: The wearable assisted capacity device apparatus of claim 4 further provided with means for causing the apparatus to produce sounds or to receive voice commands or input.

6: The wearable assisted capacity device apparatus of claim 5 further provided with a microcomputer sensor transmitter processor.

7: The wearable assisted capacity device apparatus of claim 6 further provided with a power source.

8: The wearable assisted capacity device apparatus of claim 7 further provided with a lanyard to allow the user to wear the apparatus.

9: The wearable assisted capacity device apparatus of claim 2 wherein the apparatus is housed in a watch.

10: The wearable assisted capacity device apparatus of claim 9 further provided with means for causing the apparatus to vibrate.

11: The wearable assisted capacity device apparatus of claim 10 further provided with means for causing the apparatus to produce sounds or to receive voice commands or input and further wherein the apparatus is provided with means for allowing the apparatus to receive visual input dietary information to assist in the calculation of data regarding the user's food intake.

12: The wearable assisted capacity device apparatus of claim 11 further provided with a watchband.

13: The wearable assisted capacity device apparatus of claim 12 further provided with a bracelet.

14: The wearable assisted capacity device apparatus of claim 1 wherein the appliance comprises an electronic tablet.

15: The wearable assisted capacity device apparatus of claim 1 wherein the appliance comprises a smart phone.

16: The wearable assisted capacity device apparatus of claim 1 further provided means for sensing the user's body temperature.

17: The wearable assisted capacity device apparatus of claim 1 further provided means for sensing the user's heart rate.

18: The wearable assisted capacity device apparatus of claim 1 further provided means for sensing the user's respiratory rate.

19: A wearable assisted capacity device apparatus for use by a user, comprising a lanyard or necklace and wherein
the lanyard or necklace is provided with the apparatus which can electronically communicating, by wireless signals with an electronic appliance in the form of an electronic tablet and/or smart phone device and wherein
the apparatus is provided with a button which can be activated by the user and wherein
the apparatus which can create and transmits electronic signals, via a wireless signal transmitter imbedded in the device in the form of a microcomputer transmitter sensor processor and further wherein upon activation the microcomputer sensor transmitter processor sends a wireless signal by either a wireless technology standard for exchanging data over short distances and further wherein the wireless signal can be transmitted to a medical alert staff desk, nursing station or emergency office or facility and further wherein the wireless signal is transmittable to a mobile electronic appliance either in the form of a smart phone, or an electronic tablet and further wherein the electronic tablet or smart phone can send, receive, and process the wireless signal and is compatible the Android mobile operating system and/or the IPhone cellular phone mobile operating system known as Ios and further wherein
the microcomputer sensor transmitter processor can send, receive, process, and transmit data and/or signals to and from navigational positioning systems for geographically locating the user and further wherein the electronic tablet or smart phone is provided with or is accessible to computer processing application software which can process a signal data received by the apparatus and further wherein
the computer processing application software can produce visual display to inform, remind or command the user to perform a certain task and further wherein
the apparatus is powered by a battery and further wherein
the apparatus is provided with means for causing the apparatus to vibrate to act in an alarm or awakening mode and further wherein the apparatus is provided with means for causing the apparatus to produce sounds or to receive voice commands or input voice commands to inform, remind or command the user to perform a certain task and further wherein
the apparatus is provided with means for allowing the apparatus to receive visual input dietary data to assist in the calculation of data regarding the user's intake and further wherein
the apparatus is provided with means for measuring a user' temperature, heart or respiratory rate.

20: A wearable assisted capacity device apparatus for use by a user comprising
a watchband to be worn by the user wherein the apparatus is provided with a visual display produced by a computer application program software imbedded in a microcomputer sensor transmitter processor in the apparatus wherein the visual display consists of a message to inform and suggest to the user to perform a certain task and further wherein
the microcomputer sensor transmitter processor can send, receive, process, and transmit a signal to and from navigational positioning systems for geographically locating the user and further wherein
the computer processing application software can process a signal received by the apparatus and further wherein the computer processing application software can produce a visual display to inform or suggest to the user to perform a certain task and further wherein
the apparatus is powered by a battery and further wherein the apparatus is provided with means to cause the apparatus to vibrate to act in an alarm mode and further wherein
the apparatus is provided means for causing the apparatus to produce sounds or to receive voice commands or input voice commands and further wherein
the apparatus is provided with a to inform, remind, or command the user to perform a certain task and further wherein
the apparatus is provided with means for allowing the apparatus to receive visual input dietary information to assist in the calculation of data regarding the user's food intake and further wherein
further wherein the apparatus is provided with means for measuring the user's temperature, heart or respiratory rate.
